SEQUENCE LISTING (17 sequences)

SEQ ID NO: 1 - protein, 1154 aa, Artificial Sequence
  ADF3Kai-Large-NRSH1
MHHHHHHHHHHSSGSSLEVLFQGPARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVSRARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSIL

SEQ ID NO: 2 - protein, 660 aa, Artificial Sequence
  ADF3Kai
MHHHHHHHHHHSSGSSLEVLFQGPARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 3 - protein, 1183 aa, Artificial Sequence
  ADF3Kai-Large
MHHHHHHHHHHSSGSSLEVLFQGPARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVSRARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 4 - protein, 24 aa, Artificial Sequence
  His tag and start codon
MHHHHHHHHHHSSGSSLEVLFQGP

SEQ ID NO: 5 - DNA, 1983 bp, Artificial Sequence
  ADF3Kai
atgcatcaccatcatcatcatcaccaccaccattcctcgggctcatccttggaagtgttatttcaaggaccagcacgagccggttcgggacaacaagggcctggccagcagggcccaggtcaacaagggccaggacagcagggtccttatgggcccggcgcaagcgcagcagctgcggccgctggtggctatggtcctggctccggtcaacagggcccttcgcaacaaggtcccgggcagcaaggtcctggtggccagggtccctacgggccgggggcgagtgcggcagcagccgctgcaggcggttatggtccaggaagcggacagcaaggtccgggaggtcaaggtccgtatggcccaggctctagcgcggctgccgctgccgcgggtggcaacggaccagggagcggacaacagggcgcgggacaacagggtccaggacagcaaggcccaggggcgtcggcggctgcagcggcggccggaggctatggacccggctcaggacaacagggaccgggtcaacaaggacccggtggccaaggcccctatggcccgggcgccagcgcggccgcagccgccgcgggcgggtacggccccggtagcggccagggaccaggtcagcaggggccaggaggtcagggcccatacggtccgggcgcatccgcggcggcggcagcggcaggtggctacggtcccggaagcggccaacaggggccagggcaacaaggaccaggacaacaaggtcctgggggccaaggaccgtatggaccaggagcatcagctgcagccgcggcagctggcggttacggtccaggctacggccagcagggtccgggtcagcagggaccgggaggccaggggccttatggccctggcgcttccgcagccagtgccgcttctggaggatacgggccgggaagcggtcagcaaggccctggccaacaaggacctggaggccaagggccctacggcccaggagcctcggcagccgcagctgccgcaggtgggtatgggccaggtagcgggcaacaagggccgggtcagcaaggaccggggcaacagggacctgggcagcaaggacccgggggtcaaggcccgtacggacctggtgcgtctgcagctgctgctgcggctggtggatatggtccgggatcggggcagcagggtcccggtcagcagggccctggtcagcaagggccaggccaacagggacccggacaacaaggcccgggtcaacagggtcctggacagcaggggccgggccaacaaggccctgggcaacagggtccggggggacagggggcctatgggcctggcgcatctgccgccgctggcgcagccggtgggtacgggcctgggtcaggtcaacaggggcctggtcaacaaggccccgggcaacagggccccggccagcaaggtccagggcagcagggcccgggacagcaagggcctggacaacaggggcccggacagcagggaccttacgggcccggtgcgagcgcagcggccgccgccgcagggggatatggccccggatcgggccagcagggaccaggccagcaaggacctggccaacagggcccggggggtcaggggccgtatggtcccggcgctgcaagtgctgcagtgtccgttggaggttacggccctcagtcttcgtctgttccggtggcgtccgcagttgcgagtagactgtcttcacctgctgcttcatcgcgagtatcgagcgctgtttcgtctcttgtctcgtcgggtcccacgaaacatgccgccctttcaaatacgatttcatctgtagtgtcccaagttagtgcaagtaacccggggttatccggatgcgacgttctcgttcaggcactcctagaagtagtatccgcgttggtgagcatcttaggcagctcctcgataggtcaaataaactatggtgcttcagcccagtatacacagatggtgggacagagcgtcgcgcaggcattggcttaa

SEQ ID NO: 6 - DNA, 3552 bp, Artificial Sequence
  ADF3Kai-Large
atgcatcaccatcatcatcatcaccaccaccattcctcgggctcatccttggaagtgttatttcaaggaccagcacgagccggttcgggacaacaagggcctggccagcagggcccaggtcaacaagggccaggacagcagggtccttatgggcccggcgcaagcgcagcagctgcggccgctggtggctatggtcctggctccggtcaacagggcccttcgcaacaaggtcccgggcagcaaggtcctggtggccagggtccctacgggccgggggcgagtgcggcagcagccgctgcaggcggttatggtccaggaagcggacagcaaggtccgggaggtcaaggtccgtatggcccaggctctagcgcggctgccgctgccgcgggtggcaacggaccagggagcggacaacagggcgcgggacaacagggtccaggacagcaaggcccaggggcgtcggcggctgcagcggcggccggaggctatggacccggctcaggacaacagggaccgggtcaacaaggacccggtggccaaggcccctatggcccgggcgccagcgcggccgcagccgccgcgggcgggtacggccccggtagcggccagggaccaggtcagcaggggccaggaggtcagggcccatacggtccgggcgcatccgcggcggcggcagcggcaggtggctacggtcccggaagcggccaacaggggccagggcaacaaggaccaggacaacaaggtcctgggggccaaggaccgtatggaccaggagcatcagctgcagccgcggcagctggcggttacggtccaggctacggccagcagggtccgggtcagcagggaccgggaggccaggggccttatggccctggcgcttccgcagccagtgccgcttctggaggatacgggccgggaagcggtcagcaaggccctggccaacaaggacctggaggccaagggccctacggcccaggagcctcggcagccgcagctgccgcaggtgggtatgggccaggtagcgggcaacaagggccgggtcagcaaggaccggggcaacagggacctgggcagcaaggacccgggggtcaaggcccgtacggacctggtgcgtctgcagctgctgctgcggctggtggatatggtccgggatcggggcagcagggtcccggtcagcagggccctggtcagcaagggccaggccaacagggacccggacaacaaggcccgggtcaacagggtcctggacagcaggggccgggccaacaaggccctgggcaacagggtccggggggacagggggcctatgggcctggcgcatctgccgccgctggcgcagccggtgggtacgggcctgggtcaggtcaacaggggcctggtcaacaaggccccgggcaacagggccccggccagcaaggtccagggcagcagggcccgggacagcaagggcctggacaacaggggcccggacagcagggaccttacgggcccggtgcgagcgcagcggccgccgccgcagggggatatggccccggatcgggccagcagggaccaggccagcaaggacctggccaacagggcccggggggtcaggggccgtatggtcccggcgctgcaagtgctgcagtgtccgtttctagagcacgagccggttcgggacaacaagggcctggccagcagggcccaggtcaacaagggccaggacagcagggtccttatgggcccggcgcaagcgcagcagctgcggccgctggtggctatggtcctggctccggtcaacagggcccttcgcaacaaggtcccgggcagcaaggtcctggtggccagggtccctacgggccgggggcgagtgcggcagcagccgctgcaggcggttatggtccaggaagcggacagcaaggtccgggaggtcaaggtccgtatggcccaggctctagcgcggctgccgctgccgcgggtggcaacggaccagggagcggacaacagggcgcgggacaacagggtccaggacagcaaggcccaggggcgtcggcggctgcagcggcggccggaggctatggacccggctcaggacaacagggaccgggtcaacaaggacccggtggccaaggcccctatggcccgggcgccagcgcggccgcagccgccgcgggcgggtacggccccggtagcggccagggaccaggtcagcaggggccaggaggtcagggcccatacggtccgggcgcatccgcggcggcggcagcggcaggtggctacggtcccggaagcggccaacaggggccagggcaacaaggaccaggacaacaaggtcctgggggccaaggaccgtatggaccaggagcatcagctgcagccgcggcagctggcggttacggtccaggctacggccagcagggtccgggtcagcagggaccgggaggccaggggccttatggccctggcgcttccgcagccagtgccgcttctggaggatacgggccgggaagcggtcagcaaggccctggccaacaaggacctggaggccaagggccctacggcccaggagcctcggcagccgcagctgccgcaggtgggtatgggccaggtagcgggcaacaagggccgggtcagcaaggaccggggcaacagggacctgggcagcaaggacccgggggtcaaggcccgtacggacctggtgcgtctgcagctgctgctgcggctggtggatatggtccgggatcggggcagcagggtcccggtcagcagggccctggtcagcaagggccaggccaacagggacccggacaacaaggcccgggtcaacagggtcctggacagcaggggccgggccaacaaggccctgggcaacagggtccggggggacagggggcctatgggcctggcgcatctgccgccgctggcgcagccggtgggtacgggcctgggtcaggtcaacaggggcctggtcaacaaggccccgggcaacagggccccggccagcaaggtccagggcagcagggcccgggacagcaagggcctggacaacaggggcccggacagcagggaccttacgggcccggtgcgagcgcagcggccgccgccgcagggggatatggccccggatcgggccagcagggaccaggccagcaaggacctggccaacagggcccggggggtcaggggccgtatggtcccggcgctgcaagtgctgcagtgtccgttggaggttacggccctcagtcttcgtctgttccggtggcgtccgcagttgcgagtagactgtcttcacctgctgcttcatcgcgagtatcgagcgctgtttcgtctcttgtctcgtcgggtcccacgaaacatgccgccctttcaaatacgatttcatctgtagtgtcccaagttagtgcaagtaacccggggttatccggatgcgacgttctcgttcaggcactcctagaagtagtatccgcgttggtgagcatcttaggcagctcctcgataggtcaaataaactatggtgcttcagcccagtatacacagatggtgggacagagcgtcgcgcaggcattggcttaa

SEQ ID NO: 7 - DNA, 3465 bp, Artificial Sequence
  ADF3Kai-Large-NRSH1
atgcatcaccatcatcatcatcaccaccaccattcctcgggctcatccttggaagtgttatttcaaggaccagcacgagccggttcgggacaacaagggcctggccagcagggcccaggtcaacaagggccaggacagcagggtccttatgggcccggcgcaagcgcagcagctgcggccgctggtggctatggtcctggctccggtcaacagggcccttcgcaacaaggtcccgggcagcaaggtcctggtggccagggtccctacgggccgggggcgagtgcggcagcagccgctgcaggcggttatggtccaggaagcggacagcaaggtccgggaggtcaaggtccgtatggcccaggctctagcgcggctgccgctgccgcgggtggcaacggaccagggagcggacaacagggcgcgggacaacagggtccaggacagcaaggcccaggggcgtcggcggctgcagcggcggccggaggctatggacccggctcaggacaacagggaccgggtcaacaaggacccggtggccaaggcccctatggcccgggcgccagcgcggccgcagccgccgcgggcgggtacggccccggtagcggccagggaccaggtcagcaggggccaggaggtcagggcccatacggtccgggcgcatccgcggcggcggcagcggcaggtggctacggtcccggaagcggccaacaggggccagggcaacaaggaccaggacaacaaggtcctgggggccaaggaccgtatggaccaggagcatcagctgcagccgcggcagctggcggttacggtccaggctacggccagcagggtccgggtcagcagggaccgggaggccaggggccttatggccctggcgcttccgcagccagtgccgcttctggaggatacgggccgggaagcggtcagcaaggccctggccaacaaggacctggaggccaagggccctacggcccaggagcctcggcagccgcagctgccgcaggtgggtatgggccaggtagcgggcaacaagggccgggtcagcaaggaccggggcaacagggacctgggcagcaaggacccgggggtcaaggcccgtacggacctggtgcgtctgcagctgctgctgcggctggtggatatggtccgggatcggggcagcagggtcccggtcagcagggccctggtcagcaagggccaggccaacagggacccggacaacaaggcccgggtcaacagggtcctggacagcaggggccgggccaacaaggccctgggcaacagggtccggggggacagggggcctatgggcctggcgcatctgccgccgctggcgcagccggtgggtacgggcctgggtcaggtcaacaggggcctggtcaacaaggccccgggcaacagggccccggccagcaaggtccagggcagcagggcccgggacagcaagggcctggacaacaggggcccggacagcagggaccttacgggcccggtgcgagcgcagcggccgccgccgcagggggatatggccccggatcgggccagcagggaccaggccagcaaggacctggccaacagggcccggggggtcaggggccgtatggtcccggcgctgcaagtgctgcagtgtccgtttctagagcacgagccggttcgggacaacaagggcctggccagcagggcccaggtcaacaagggccaggacagcagggtccttatgggcccggcgcaagcgcagcagctgcggccgctggtggctatggtcctggctccggtcaacagggcccttcgcaacaaggtcccgggcagcaaggtcctggtggccagggtccctacgggccgggggcgagtgcggcagcagccgctgcaggcggttatggtccaggaagcggacagcaaggtccgggaggtcaaggtccgtatggcccaggctctagcgcggctgccgctgccgcgggtggcaacggaccagggagcggacaacagggcgcgggacaacagggtccaggacagcaaggcccaggggcgtcggcggctgcagcggcggccggaggctatggacccggctcaggacaacagggaccgggtcaacaaggacccggtggccaaggcccctatggcccgggcgccagcgcggccgcagccgccgcgggcgggtacggccccggtagcggccagggaccaggtcagcaggggccaggaggtcagggcccatacggtccgggcgcatccgcggcggcggcagcggcaggtggctacggtcccggaagcggccaacaggggccagggcaacaaggaccaggacaacaaggtcctgggggccaaggaccgtatggaccaggagcatcagctgcagccgcggcagctggcggttacggtccaggctacggccagcagggtccgggtcagcagggaccgggaggccaggggccttatggccctggcgcttccgcagccagtgccgcttctggaggatacgggccgggaagcggtcagcaaggccctggccaacaaggacctggaggccaagggccctacggcccaggagcctcggcagccgcagctgccgcaggtgggtatgggccaggtagcgggcaacaagggccgggtcagcaaggaccggggcaacagggacctgggcagcaaggacccgggggtcaaggcccgtacggacctggtgcgtctgcagctgctgctgcggctggtggatatggtccgggatcggggcagcagggtcccggtcagcagggccctggtcagcaagggccaggccaacagggacccggacaacaaggcccgggtcaacagggtcctggacagcaggggccgggccaacaaggccctgggcaacagggtccggggggacagggggcctatgggcctggcgcatctgccgccgctggcgcagccggtgggtacgggcctgggtcaggtcaacaggggcctggtcaacaaggccccgggcaacagggccccggccagcaaggtccagggcagcagggcccgggacagcaagggcctggacaacaggggcccggacagcagggaccttacgggcccggtgcgagcgcagcggccgccgccgcagggggatatggccccggatcgggccagcagggaccaggccagcaaggacctggccaacagggcccggggggtcaggggccgtatggtcccggcgctgcaagtgctgcagtgtccgttggaggttacggccctcagtcttcgtctgttccggtggcgtccgcagttgcgagtagactgtcttcacctgctgcttcatcgcgagtatcgagcgctgtttcgtctcttgtctcgtcgggtcccacgaaacatgccgccctttcaaatacgatttcatctgtagtgtcccaagttagtgcaagtaacccggggttatccggatgcgacgttctcgttcaggcactcctagaagtagtatccgcgttggtgagcatcttataa

SEQ ID NO: 8 - protein, 601 aa, Artificial Sequence
  PRT410
MHHHHHHSSGSSGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 9 - protein, 559 aa, Artificial Sequence
  Recombinant spider silk protein Flag_92_short2
MHHHHHHHHHHSSGSSLEVLFQGPGAGGSGPGGAGPGGVGPGGSGPGGVGPGGSGPGGVGPGGSGPGGVGPGGAGGPYGPGGSGPGGAGGAGGPGGAYGPGGSYGPGGSGGPGGAGGPYGPGGEGPGGAGGPYGPGGAGGPYGPGGAGGPYGPGGEGGPYGPGGSYGPGGAGGPYGPGGPYGPGGEGPGGAGGPYGPGGVGPGGGGPGGYGPGGAGPGGYGPGGSGPGGYGPGGSGPGGYGPGGSGPGGYGPGGSGPGGYGPGGSGPGGSGPGGYGPGGSGPGGSGPGGYGPGGSGPGGYGPGGSGPGGSGPGGYGPGGSGPGGSGPGGYGPGGSGPGGFGPGGFGPGGSGPGGYGPGGSGPGGAGPGGVGPGGFGPGGAGPGGAGPGGAGPGGAGPGGAGPGGAGPGGAGPGGAGPGGAGGAGGAGGAGGSGGAGGSGGTTIIEDLDITIDGADGPITISEELTISAYYPSSRVPDMVNGIMSAMQGSGFNYQMFGNMLSQYSSGSGTCNPNNVNVLMDALLAALHCLSNHGSSSFAPSPTPAAMSAYSNSVGRMFAY

SEQ ID NO: 10 - protein, 252 aa, Artificial Sequence
  Collagen-type4-Kai
MHHHHHHSSGSSKDGVPGFPGSEGVKGNRGFPGLMGEDGIKGQKGDIGPPGFRGPTEYYDTYQEKGDEGTPGPPGPRGARGPQGPSGPPGVPGSPGSSRPGLRGAPGWPGLKGSKGERGRPGKDAMGTPGSPGCAGSPGLPGSPGPPGPPGDIVFRKGPPGDHGLPGYLGSPGIPGVDGPKGEPGLLCTQCPYIPGPPGLPGLPGLHGVKGIPGRQGAAGLKGSPGSPGNTGLPGFPGFPGAQGDPGLKGEK

SEQ ID NO: 11 - protein, 310 aa, Artificial Sequence
  Resilin-Kai
MHHHHHHPEPPVNSYLPPSDSYGAPGQSGPGGRPSDSYGAPGGGNGGRPSDSYGAPGQGQGQGQGQGGYAGKPSDSYGAPGGGDGNGGRPSSSYGAPGGGNGGRPSDTYGAPGGGNGGRPSDTYGAPGGGGNGNGGRPSSSYGAPGQGQGNGNGGRPSSSYGAPGGGNGGRPSDTYGAPGGGNGGRPSDTYGAPGGGNNGGRPSSSYGAPGGGNGGRPSDTYGAPGGGNGNGSGGRPSSSYGAPGQGQGGFGGRPSDSYGAPGQNQKPSDSYGAPGSGNGNGGRPSSSYGAPGSGPGGRPSDSYGPPASG

SEQ ID NO: 12 - protein, 282 aa, Artificial Sequence
  elastin short
MHHHHHHSSGSSLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVG

SEQ ID NO: 13 - protein, 468 aa, Artificial Sequence
  type I keratin 26
MSFRLSGVSRRLCSQAGTGRLTGGRTGFRAGNVCSGLGAGSSFSGPLGSVSSKGSFSHGGGGLGSGVCTGFLENEHGLLPGNEKVTLQNLNDRLASYLDHVCTLEEANADLEQKIKGWYEKYGPGSGRQLAHDYSKYFSVTEDLKRQIISVTTCNASIVLQNENARLTADDFRLKCENELALHQSVEADINGLHRVMDELTLCTSDLEMQCEALSEELTYLKKNHQEEMKVMQGAARGNVNVEINAAPGVDLTVLLNNMRAEYEDLAEQNHEDAEAWFSEKSTSLHQQISDDAGAAMAARNELMELKRNLQTLEIELQSLLAMKHSYECSLAETESNYCHQLQQIQEQIGAMEDQLQQIRMETEGQKLEHERLLDVKIFLEKEIEMYCKLIDGEGRKSKSTCYKSEGRGPKNSENQVKDSKEEAVVKTVVGELDQLGSVLSLRVHSVEEKSSKISNITMEQRLPSKVP

SEQ ID NO: 14 - DNA, 20 bp, Artificial Sequence
  T7 promoter primer
taatacgactcactataggg

SEQ ID NO: 15 - DNA, 27 bp, Artificial Sequence
  Rep Xba I primer
tctagaaacggacactgcagcacttgc

SEQ ID NO: 16 - DNA, 28 bp, Artificial Sequence
  Xba I Rep primer
tctagagcacgagccggttcgggacaac

SEQ ID NO: 17 - DNA, 19 bp, Artificial Sequence
  T7 terminator primer
gctagttattgctcagcgg

(19) United States
(12) Patent Application Publication
(10) Pub. No.: US 2019/0002644 A1
(43) Pub. Date: Jan. 3, 2019

(54) Method for Producing Polymer Aggregate

(71) Applicant: SPIBER INC., Tsuruoka-shi, Yamagata (JP)

(72) Inventors: Yoshinori OHTA, Yamagata (JP); Hiroaki SUZUMURA, Yamagata (JP)

(73) Assignee: Spiber Inc., Tsuruoka-shi, Yamagata (JP)

(21) Appl. No.: 16/065,391

(22) PCT Filed: Dec. 21, 2016
(86) PCT No.: PCT/JP2016/088205

§ 371 Date: Jun. 22, 2018

(30) Foreign Application Priority Data
Dec. 25, 2015 (JP) 2015-254210

(51) Int. Cl. C08J 3/07; C07K 14/435; D01F 4/02

(52) U.S. Cl. CPC C08J 3/07; C08J 2389/00; D01F 4/02; C07K 14/43518

(57) ABSTRACT

The present invention relates to a method for producing a polymer aggregate, the method including: a step for bringing a polymer solution containing a polymer substance and cations for gel formation into contact with an anion solution containing anions for gel formation to form a gel at an interface between the polymer solution and the anion solution; and a step for causing the polymer substance to aggregate inside the gel.

### SEQUENCE LISTING SUBMISSION VIA EFS-WEB

A computer readable text file, entitled “SequenceListing.txt,” created on or about Jun. 21, 2018 with a file size of about 60 kb contains the sequence listing for this application and is hereby incorporated by reference in its entirety.

### TECHNICAL FIELD

The present invention relates to a method for producing a polymer aggregate.

### BACKGROUND ART

Spiders' threads are also referred to as spider silks and known to be produced by biosynthetic technologies using natural spider silks as a starting material. Polymer aggregates obtained by molding spider silk proteins in various forms are known. For example, Patent Literature 1 discloses a film using a spider silk protein. In addition, Patent Literature 2 discloses polypeptide particles derived from a spider silk protein.

### CITATION LIST

### Patent Literature

Patent Literature 1: Japanese Unexamined Patent Publication No. 2008-507260

Patent Literature 2: WO2014/61043

### SUMMARY OF INVENTION

### Problems to be Solved by the Invention

However, a protein is easily dissolved in water or other solvents, and even when the protein is extruded in a predetermined form to a coagulation liquid, the protein is dissolved in the coagulation liquid, and thus it is difficult to obtain a molded article in a fibrous form, a film form, or the like in high yield.

In this regard, an object of the present invention is to provide a method for producing a polymer aggregate by which polymer aggregates in various forms can be easily obtained.

### Means for Solving the Problems

The present invention provides a method for producing a polymer aggregate, the method including: a step for bringing a polymer solution containing a polymer substance and cations for gel formation into contact with an anion solution containing anions for gel formation to form a gel at an interface between the polymer solution and the anion solution; and a step for causing the polymer substance to aggregate inside the gel.

In the production method of the present invention, the gel is formed at the interface between the polymer solution and the anion solution by the cations for gel formation and the anions for gel formation. According to this, a solution containing the polymer substance is enclosed inside the formed gel. At this time, the gel functions as a dialysis membrane. Small molecules in the polymer solution and the anion solution (for example, a solvent, a salt, and the like) move through the gel, whereas the polymer substance remains inside the gel and aggregates to form a polymer aggregate. In the production method, the form of the polymer aggregate thus obtained depends on the form of the gel thus formed. Since the gel is formed by contact between the polymer solution and the anion solution, the gel can be formed in various forms such as a fiber, a film, a hollow pipe, and a bead, by simple operation. Thus, according to the production method of the present invention, polymer aggregates in various forms can be easily obtained.

The cations for gel formation may be at least one selected from multivalent cations or hydrogen ions. In addition, the anions for gel formation may be alginate ions.

The anion solution preferably further contains an aggregation promoter for the polymer substance. According to this, a polymer aggregate can be produced in a shorter time.

The polymer solution may further contain a dissolution promoter for the polymer substance.

In the production method of the present invention, since the polymer substance does not move through the gel, a polymer aggregate can be produced in a water system. Thus, solvents of the polymer solution and the anion solution may be water. Since the polymer aggregate can be produced in the water system, processes such as waste liquid treatment can be simplified, and there is no risk that non-aqueous solvent is mixed into the polymer aggregate thus formed. Thus, there is also an advantage that application of the polymer aggregate can be easily widened.

The production method of the present invention is based on the action that the gel functions as a dialysis membrane and the polymer substance is aggregated inside the gel, and thus the type of the polymer substance is not particularly limited. However, typically, the polymer substance is preferably a protein. In addition, the type of the protein is not particularly limited, but for example, may be a polypeptide derived from a spider silk protein.

### Effects of the Invention

According to the present invention, it is possible to provide a method for producing a polymer aggregate by which polymer aggregates in various forms can be easily obtained. In the production method of the present invention, the form of the polymer aggregate thus obtained depends on the form of the gel thus obtained. Since the gel is formed by contact between the polymer solution and the anion solution, the gel can be formed in various forms such as a fiber, a film, a hollow pipe, and a bead, by simple operation.

### BRIEF DESCRIPTION OF DRAWINGS

FIG. 1 is an explanatory diagram illustrating a production method according to an embodiment. FIG. 1, drawing (A) is an explanatory diagram illustrating a step for extruding a polymer solution (dope) into an anion solution (coagulation liquid). FIG. 1, drawing (B) is an explanatory diagram illustrating a step for immersing a polymer aggregate covered with a gel in water and further causing the polymer aggregate to aggregate.

FIG. 2 is an explanatory diagram illustrating a production method according to an embodiment.

FIG. 3 is an explanatory diagram illustrating a production method according to an embodiment.

FIG. 4, drawing (A) is an explanatory diagram illustrating a production method according to an embodiment. FIG. 4, drawing (B) is an enlarged view of a part surrounded by A in FIG. 4, drawing (A).

FIG. 5 is a schematic cross-sectional view illustrating a coagulation liquid-gel-dope interface in a production method according to an embodiment.

FIG. 6 is a production process diagram according to an embodiment.

FIG. 7 is a photograph showing a polymer aggregate obtained in Example 1 (in which spider silk proteins aggregate to be formed in a fiber form) and a trace drawing which traces the photograph. FIG. 7, drawing (A) is a photograph showing a polymer aggregate formed in a gel. FIG. 7, drawing (B) is a photograph showing the polymer aggregate from which the gel is removed. FIG. 7, drawing (C) is a trace drawing which traces the photograph of FIG. 7, drawing (A). FIG. 7, drawing (D) is a trace drawing which traces the photograph of FIG. 7, drawing (B).

FIG. 8 is a photograph showing a polymer aggregate obtained in Example 2 (in which spider silk proteins aggregate to be formed in a fiber form) and a trace drawing which traces the photograph. FIG. 8, drawing (A) is a photograph showing a polymer aggregate formed in a gel. FIG. 8, drawing (B) is a photograph showing the polymer aggregate from which the gel is removed. FIG. 8, drawing (C) is a trace drawing which traces the photograph of FIG. 8, drawing (A). FIG. 8, drawing (D) is a trace drawing which traces the photograph of FIG. 8, drawing (B).

FIG. 9 is a photograph showing a polymer aggregate obtained in Example 3 (in which silk proteins aggregate to be formed in a fiber form) and a trace drawing which traces the photograph. FIG. 9, drawing (A) is a photograph showing a polymer aggregate formed in a gel. FIG. 9, drawing (B) is a trace drawing which traces the photograph of FIG. 9, drawing (A).

FIG. 10 is a photograph showing a polymer aggregate obtained in Example 4 (in which silk proteins aggregate to be formed in a fiber form) and a trace drawing which traces the photograph. FIG. 10, drawing A is a photograph showing a polymer aggregate formed in a gel. FIG. 10, drawing (B) is a trace drawing which traces the photograph of FIG. 10, drawing (A).

FIG. 11 is a photograph showing a polymer aggregate obtained in Example 6 (in which gelatin proteins aggregate to be formed in a fiber form) and a trace drawing which traces the photograph. FIG. 11, drawing (A) is a photograph showing a polymer aggregate formed in a gel. FIG. 11, drawing (B) is a trace drawing which traces the photograph of FIG. 11, drawing (A).

### EMBODIMENTS FOR CARRYING OUT THE INVENTION

Hereinafter, preferred embodiments of the present invention will be described in detail with reference to the drawings in some cases; however, the present invention is not limited to the following embodiments. Incidentally, in the drawings, the same or equivalent parts are attached with the same signs, and duplicate descriptions are appropriately omitted.

A method for producing a polymer aggregate according to the present invention includes a step for bringing a polymer solution containing a polymer substance and cations for gel formation into contact with an anion solution containing anions for gel formation to form a gel at an interface between the polymer solution and the anion solution (hereinafter, also referred to as “gel formation step”), and a step for causing the polymer substance to aggregate inside the gel (hereinafter, also referred to as “aggregation step”).

(Polymer Solution)

The polymer substance serving as a raw material is not particularly limited, and an arbitrary polymer substance can be used. Specific examples of the polymer substance include biological polymers such as proteins, nucleic acids, lipids, and polysaccharides (for example, cellulose, starch, and the like) and synthetic polymers such as synthetic resins (for example, polyvinyl chloride, polyethylene, phenolic resin, and the like), silicon resins (for example, silicon rubber, and the like), synthetic fibers (for example, nylon, vinylon, polyester, polyethylene terephthalate, and the like), and synthetic rubbers (for example, butadiene rubber, isoprene rubber, and the like).

Since biological polymers such as proteins have a room for improvement particularly in the production process of the polymer aggregate, the production method according to the present invention is suitably applied to the biological polymers such as proteins. The type of protein is not limited, and an arbitrary protein can be applied. The protein serving as a raw material may be produced using microorganisms or the like by genetic engineering technology, may be produced by synthesis, or may be produced by purifying a naturally derived protein.

The protein is preferably a structural protein. The structural protein is a protein having a role of constructing a living structure and is different from a functional protein such as enzyme, hormone, or antibody. Examples of the structural protein may include natural structural proteins such as fibroin, collagen, resilin, elastin, and keratin that occur in nature. As naturally occurring fibroin, fibroins produced by insects and spiders are known. In this embodiment, the structural protein preferably includes spider silk proteins. Incidentally, among structural proteins, at least one selected from a polypeptide (spider silk fibroin) derived from a spider silk protein, a silk protein (silken thread, silk fibroin, or the like), gelatin, collagen, and elastin is preferable. These proteins are easily formed in various forms including a fiber.

Examples of fibroin produced by insects include silk proteins produced by silkworms such as Bombyx mori, Bombyx mandarina, Antheraea yamamai, Anteraea pernyi, Eriogyna pyretorum, Pilosamia Cynthia ricini, Sarnia cynthia, Caligura japonica, Antheraea mylitta, and Antheraea assama; and hornet silk proteins discharged from larvae of Vespa simillima xanthoptera.

As a more specific example of fibroin produced by insects, for example, Bombyx mori fibroin L chain (GenBank Accession No.: M76430 (base sequence), AAA27840.1 (amino acid sequence)) is exemplified.

The silken thread is a fiber obtained from a cocoon produced by a silkworm, which is a larva of Bombyx mori (cocoon filament). Two fibroins are covered with a sticky substance (sericin) to form one cocoon filament. The fibroin is composed of a plurality of fibrils, and the outer side of the fibroin is covered with the sericin having four layers, thereby configuring one cocoon filament.

The silk fibroin is obtained by using a natural or domesticated cocoon or a used or waste silk cloth as a raw material, removing sericin covering the fibroin and other substances such as fat therefrom, and purifying the fibroin. The silk fibroin is preferably silk fibroin obtained by forming the purified fibroin into freeze-dried powder.

The spider silk fibroin is fibroin produced by spiders. A spider has a maximum of seven types of silk gland, and the silk glands produce fibroins (spider silk proteins) each having a different property, respectively. The spider silk proteins are designated, according to organs of origins thereof, as a major ampullate spider protein (MaSp) having high toughness, a minor ampullate spider protein (MiSp) having a high level of extension force, and flagelliform (Flag), tubuliform, aggregate, aciniform, and pyriform spider silk proteins.

Examples of the fibroin produced by spiders include spider silk proteins produced by spiders belonging to the genus Araneus such as Araneus ventricosus, Araneus diadematus, Araneus quadratus, Araneus pentagrammicus, and Araneus nojimai, spiders belonging to the genus Neoscona such as Neoscona scylla, Neoscona nautica, Neoscona adianta, and Neoscona scylloides, spiders belonging to the genus Pronus such as Pronous minutus, spiders belonging to the genus Cyrtarachne such as Cyrtarachne bufo and Cyrtarachne inaequalis, spiders belonging to the genus Gasteracantha such as Gasteracantha kuhlii and Gasteracantha mammosa, spiders belonging to the genus Ordgarius such as Ordgarius hobsoni and Ordgarius sexspinosus, spiders belonging to the genus Argiope such as Argiope amoena, Argiope minuta, and Argiope bruennichi, spiders belonging to the genus Arachnura such as Arachnura logio, spiders belonging to the genus Acusilas such as Acusilas coccineus, spiders belonging to the genus Cytophora such as Cyrtophora moluccensis, Cyptoployd extMerragtica, and Cyrtophora unicolor, spiders belonging to the genus Poltys such as Poltys illepidus, spiders belonging to the genus Cyclosa such as Cyclosa octotuberculata, Cyclosa sedeculata, Cyclosa vallata, and Cyclosa atrata, and spiders belonging to the genus Chorizopes such as Chorizopes nipponicus; and spider silk proteins produced by spiders belonging to the Tetragnathidae family including spiders belonging to the genus Tetragnatha such as Tetragnatha praedonia, Tetragnatha maxillosa, Tetragnatha extensa, and Tetragnatha squamata, spiders belonging to the genus Leucauge such as Leucauge magnifica, Leucauge blanda, and Leucauge subblanda, spiders belonging to the genus Nephila such as Nephila clavata and Nephila pilipes, spiders belonging to the genus Menosira such as Menosira ornata, spiders belonging to the genus Dyschiriognatha such as Pachygnatha tenera, spiders belonging to the genus Latrodectus such as Latrodectus mactans, Latrodectus hasseltii, Latrodectus geometricus, and Latrodectus tredecimguttatus, spiders belonging to the genus Euprosthenops, and the like. Examples of the spider silk proteins include MaSp (MaSp1 and MaSp2), dragline proteins such as ADF (ADF3 and ADF4), and MiSp (MiSp1 and MiSp2).

More specific examples of the fibroin produced by spiders include fibroin-3 (adf-3) [derived from Araneus diadematus] (GenBank Accession No.: AAC47010 (amino acid sequence), U47855 (base sequence)), fibroin-4 (adf-4) [derived from Araneus diadematus] (GenBank Accession No.: AAC47011 (amino acid sequence), U47856 (base sequence)), dragline silk protein spidroin 1 [derived from Nephila clavipes] (GenBank Accession No.: AAC04504 (amino acid sequence), U37520 (base sequence)), major ampullate spidroin 1 [derived from Latrodectus hesperus] (GenBank Accession No.: ABR68856 (amino acid sequence), EF595246 (base sequence)), dragline silk protein spidroin 2 [derived from Nephila clavata] (GenBank Accession No.: AAL32472 (amino acid sequence), AF441245 (base sequence)), major ampullate spidroin 1 [derived from Euprosthenops australis] (GenBank Accession No.: CAJ00428 (amino acid sequence), AJ973155 (base sequence)), and major ampullate spidroin 2 [Euprosthenops australis] (GenBank Accession No.: CAM32249.1 (amino acid sequence), AM490169 (base sequence)), minor ampullate silk protein 1 [Nephila clavipes] (GenBank Accession No.: AAC14589.1 (amino acid sequence)), minor ampullate silk protein 2 [Nephila clavipes] (GenBank Accession No.: AAC14591.1 (amino acid sequence)), and minor ampullate spidroin-like protein [Nephilengys cruentata] (GenBank Accession No.: ABR37278.1 (amino acid sequence).

More specific examples of the naturally derived fibroin may further include fibroins whose sequence information is registered in NCBI GenBank. For example, this can be confirmed by extracting, from sequences including INV as DIVISION of the sequence information registered in NCBI GenBank, sequences described as a keyword of spidroin, ampullate, fibroin, “silk and polypeptide,” or “silk and protein” in DEFINITION, and sequences described by character strings of specific products from CDS and specific character strings in TISSUE TYPE from SOURCE.

The protein may be a polypeptide derived from the natural protein, that is, a recombinant polypeptide. For example, the recombinant fibroin is produced in several foreign protein production systems, and as the production method therefor, transgenic goats, transgenic silkworms, or recombinant plants or mammalian cells are used.

The recombinant fibroin can also be obtained by designing an amino acid sequence of naturally derived fibroin or an amino acid sequence, which corresponds to modification of an amino acid sequence corresponding to substitution, deletion, insertion and/or addition of one or plural amino acid residues to an amino acid sequence of naturally derived fibroin, and chemically synthesizing a nucleic acid encoding the designed amino acid sequence.

The recombinant polypeptide of major dragline silk protein can be represented, for example, as a protein containing a domain sequence represented by Formula 1: [(A)ₙ motif-REP]. (here, in Formula 1, (A)ₙ motif represents an amino acid sequence composed of 4 to 20 amino acid residues, and the number of alanine residues to the number of whole amino acid residues in the (A)ₙ motif is 70% or more; REP represents an amino acid sequence composed of 10 to 200 amino acid residues; m represents an integer of 5 to 300; a plurality of (A)ₙ motifs may be the same or different amino acid sequence from each other; and a plurality of REPs may be the same or different amino acid sequence from each other.). Specifically, proteins containing amino acid sequences represented by SEQ ID NOs: 1 to 3 and SEQ ID NOs: 8 and 9 can be exemplified.

Examples of the recombinant polypeptide of collagen may include proteins containing a domain sequence represented by Formula 2: [REP2]ₒ (here, in Formula 2, o represents an integer of 5 to 300; REP2 represents an amino acid sequence composed of Gly-X-Y; X and Y represent an arbitrary amino acid residue other than Gly; and a plurality of REP2s may be the same or different amino acid sequence from each other.). Specifically, a protein containing an amino acid sequence represented by SEQ ID NO: 10 can be exemplified. The amino acid sequence represented by SEQ ID NO: 10 is an amino acid sequence obtained by adding an amino acid sequence represented by SEQ ID NO: 4 to the N-terminal of a partial sequence of human collagen type 4 obtained from the NCBI database (NCBI Genbank Accession No.: CAA56335.1, GI: 3702452), specifically, an amino acid sequence thereof from the 301st residue to the 540th residue that corresponds to repetitive sections and motifs in the partial sequence of human collagen type 4.

Examples of the recombinant polypeptide of resilin may include proteins containing a domain sequence represented by Formula 3: [REP3]ₚ (here, in Formula 3, p represents an integer of 4 to 300; REP3 represents an amino acid sequence composed of Ser-J-J-Tyr-Gly-U-Pro; J represents an arbitrary amino acid residue, and particularly, is preferably an amino acid residue selected from the group consisting of Asp, Ser, and Thr; U represents an arbitrary amino acid residue, and particularly, is preferably an amino acid residue selected from the group consisting of Pro, Ala, Thr, and Ser; and a plurality of REP3s may be the same or different amino acid sequence from each other.). Specifically, a protein containing an amino acid sequence represented by SEQ ID NO: 11 can be exemplified. The amino acid sequence represented by SEQ ID NO: 11 is an amino acid sequence obtained as follows: in an amino acid sequence of resilin (NCBI Genbank Accession No.: NP 611157, GI: 24654243), the 87th residue Thr and the 95th residue Asn are substituted with Ser and Asp, respectively, and an amino acid sequence represented by SEQ ID NO: 4 is added to the N-terminal of an amino acid sequence from the 19th residue to the 321st residue of the substituted sequence.

Examples of the recombinant polypeptide of elastin may include proteins containing amino acid sequences of NCBI Genbank Accession Nos.: AAC98395 (human), 147076 (sheep), NP786966 (bovine), and the like. Specifically, a protein containing an amino acid sequence represented by SEQ ID NO: 12 can be exemplified. The amino acid sequence represented by SEQ ID NO: 12 is amino acid sequence obtained by adding an amino acid sequence represented by SEQ ID NO: 4 to the N-terminal of an amino acid sequence from the 121st residue to the 390th residue of the amino acid sequence of NCBI Genbank Accession No.: AAC98395.

Examples of the recombinant polypeptide of keratin may include Capra hircus type I keratin. Specifically, a protein containing an amino acid sequence represented by SEQ ID NO: 13 (amino acid sequence of NCBI Genbank Accession No.: ACY30466) can be exemplified.

The recombinant polypeptide may be a recombinant fibroin containing an amino acid sequence having a sequence identity of 90% or more with the amino acid sequence of the above-described fibroin.

The aforementioned recombinant fibroin may contain a tag sequence at either or both of the N-terminal and the C-terminal According to this, isolation, immobilization, detection, visualization, and the like of the recombinant fibroin are enabled.

As the tag sequence, for example, an affinity tag using specific affinity (affinity) with other molecules can be exemplified. As a specific example of the affinity tag, a histidine tag (His tag) can be exemplified. The His tag is a short peptide containing 4 to 10 histidine residues arranged and has a property that specifically binds to metal ions such as nickel. Thus, the His tag can be used for isolation of recombinant fibroin by chelating metal chromatography. As a specific example of the tag sequence, for example, an amino acid sequence represented by SEQ ID NO: 4 is exemplified.

Further, a tag sequence such as glutathione-S-transferase (GST) specifically binding to glutathione or maltose-binding protein (MBP) specifically binding to maltose can also be used.

Furthermore, an “epitope tag” using antigen-antibody reaction can also be used. By adding peptide (epitope) exhibiting antigenicity as a tag sequence, an antigen can be bound to the epitope. Examples of the epitope tag may include a HA (peptide sequence of hemagglutinin of influenza virus) tag, a myc tag, and a FLAG tag. By using the epitope tag, recombinant fibroin can be easily purified with high specificity.

Further, one obtained by isolating a tag sequence with a specific protease can also be used. By performing protease treatment to a protein adsorbed through the tag sequence, recombinant fibroin from which the tag sequence is isolated can also be collected.

A method for producing a protein by the genetic recombination technology will be described below. Such a protein can be produced, for example, by expressing a nucleic acid by a host transformed by an expression vector which has a nucleic acid sequence encoding the protein and one or a plurality of regulatory sequences operably linked to the nucleic acid sequence.

A method for producing a gene encoding a protein is not particularly limited. For example, a gene can be produced by a method of using a gene encoding a natural structural protein and amplifying and cloning the gene by polymerase chain reaction (PCR) or the like or can be produced by chemical synthesis. The method of chemically synthesizing a gene is also not particularly limited, and for example, a gene can be chemically synthesized by a method of linking oligonucleotide, which is automatically synthesized by AKTA oligopilot plus 10/100 (GE healthcare Japan, Ltd.) or the like, by PCR or the like based on amino acid sequence information of structural protein obtained from NCBI web database or the like. At this time, in order to facilitate purification and confirmation of a protein, a gene encoding a protein, which is composed of an amino acid sequence obtained by adding an amino acid sequence composed of the start codon and the His 10-tag to the N-terminal of the amino acid sequence, may be synthesized.

The regulatory sequence is a sequence regulating expression of a recombinant protein in a host (for example, a promoter, an enhancer, a ribosome binding sequence, a transcription termination sequence, or the like), and can be appropriately selected according to the type of host. As a promoter, an inducible promoter which functions in host cells and is capable of inducing expression of a target protein, may be used. The inducible promoter is a promoter which can control transcription in the presence of an inducing substance (expression inducer), in the absence of a repressor molecule, or by a physical factor such as an increase or decrease in temperature, osmotic pressure or pH value.

As for the type of the expression vector, a plasmid vector, a virus vector, a cosmid vector, a fosmid vector, an artificial chromosome vector, and the like can be appropriately selected depending on the type of the host. As the expression vector, those capable of autonomous replication or integration into the chromosome in the host and containing a promoter at a position where a nucleic acid encoding a target protein can be transcripted are preferably used.

As the host, any of prokaryotes and eukaryotes such as yeasts, filamentous fungi, insect cells, animal cells, and plant cells can be preferably used.

Preferable examples of prokaryotes may include bacteria belonging to the genera Escherichia, Brevibacillus, Serratia, Bacillus, Microbacterium, Brevibacterium, Corynebacterium, Pseudomonas, and the like.

Examples of a vector, which introduces a nucleic acid encoding a protein, may include pBTrp2 (manufactured by Boehringer Mannheim GmbH), pGEX (manufactured by Pharmacia), and pUC18, pBluescriptII, pSupex, pET22b, pCold, pUB110, and pNCO2 (Japanese Unexamined Patent Publication No. 2002-238569).

Examples of hosts of eukaryotes may include yeasts and filamentous fungi (fungi and the like).

Examples of the yeasts may include yeasts belonging to the genera Saccharomyces, Pichia, Schizosaccharomyces, and the like. Examples of the filamentous fungi may include filamentous fungi belonging to the genera Aspergillus, Penicillium, Trichoderma, and the like.

Examples of the vector may include YEP13 (ATCC37115) and YEp24 (ATCC37051).

As a method of introducing the expression vector into the host cell, any method can be used as long as it is a method of introducing DNA into the host cell. Examples of the introduction method may include a method using calcium ions [Proc. Natl. Acad. Sci. USA, 69, 2110 (1972)], an electroporation method, a spheroplast method, a protoplast method, a lithium acetate method, and a competent method.

As the method of expressing a nucleic acid by a host transformed by an expression vector, other than the direct expression, production by secretion, expression of fusion protein, or the like can be performed according to the method described in Molecular Cloning, 2nd edition, or the like.

A protein can be produced, for example, by culturing a host transformed by an expression vector in an incubation medium, growing and accumulating the protein in the incubation medium, and collecting the protein from the incubation medium. The method of culturing the host in the incubation medium can be performed according to a method generally used in culturing of a host.

In a case where a host is prokaryotes such as Escherichia coli or eukaryotes such as yeast, as an incubation medium of the host, either natural or synthetic culture medium may be used as long as it contains a carbon source, a nitrogen source, inorganic salts, and the like that may be assimilated by the host, and culturing of the host is efficiently carried out.

The carbon source may be any that may be assimilated by the host, and for example, glucose, fructose, sucrose, molasses containing these, carbohydrates such as starch and starch hydrolysate, organic acids such as acetic acid and propionic acid, and alcohols such as ethanol and propanol can be used.

As the nitrogen source, for example, ammonia, an ammonium salt of inorganic acids or organic acids such as ammonium chloride, ammonium sulfate, ammonium acetate, and ammonium phosphate, other nitrogen-containing compounds, as well as peptone, meat extract, yeast extract, corn steep liquor, casein hydrolysate, soybean cake and soybean cake hydrolysate, and various zymocytes and digests thereof can be used.

As inorganic salts, for example, monopotassium phosphate, dipotassium phosphate, magnesium phosphate, magnesium sulfate, sodium chloride, ferrous sulfate, manganese sulfate, copper sulfate, and calcium carbonate can be used.

The culturing of prokaryotes such as Escherichia coli or eukaryote such as yeast can be carried out, for example, under aerobic conditions such as shaking culture or deep aeration stirring culture. The culture temperature is, for example, 15 to 40° C. The culture time is typically 16 hours to 7 days. The pH of the incubation medium during cultivating is preferably maintained to 3.0 to 9.0. The adjustment of pH of the incubation medium can be performed using an inorganic acid, an organic acid, an alkali solution, urea, calcium carbonate, ammonia, or the like.

Further, antibiotic such as ampicillin and tetracycline may also be added to an incubation medium as necessary during cultivating. When cultivating a microorganism transformed with an expression vector using an inducible promoter as the promoter, an inducer may be added to the culture medium as necessary. For example, when cultivating a microorganism transformed with an expression vector using a lac promoter, isopropyl-β-D-thiogalactopyranoside or the like may be added, and when cultivating a microorganism transformed with an expression vector using a trp promoter, indoleacrylic acid or the like may be used.

Isolation and purification of the protein can be performed by a method generally used. For example, in a case where a protein is expressed in a dissolved state within the cell, after the culturing is complete, host cells are recovered by centrifugation, suspended in an aqueous buffer solution, and then the host cells are disrupted with a sonicator, a french press, Manton-Gaulin homogenizer, Dynomill, or the like to obtain a cell-free extract. From the supernatant obtained by centrifuging the cell-free extract, a method generally used for isolation and purification of the protein, that is, methods such as a solvent extraction method, a salt precipitation method by ammonium sulfate or the like, a desalting method, a precipitation method by an organic solvent, diethylaminoethyl (DEAE)-Sepharose, an anion exchange chromatography method using a resin such as DIAION HPA-75 (manufactured by Mitsubishi Chemical Corporation), a cation exchange chromatography method using a resin such as S-Sepharose FF (manufactured by Pharmacia), a hydrophobic chromatography method using a resin such as butyl Sepharose or phenyl Sepharose, a gel filtration method using a molecular sieve, an affinity chromatography method, a chromatofocusing method, and an electrophoresis method such as isoelectric electrophoresis are used alone or in combination, and a purified preparation can be obtained.

Further, in a case where the protein is expressed by forming an insoluble form within cells, similarly, the host cells are disrupted after recovery and centrifuged to recover the insoluble form of the modified fibroin as a precipitated fraction. The insoluble form of the modified fibroin thus recovered can be dissolved with a protein denaturant. After the operation, a purified preparation of the modified fibroin can be obtained by the isolation and purification method similar to the above.

In a case where a protein is secreted outside the cell, the protein can be recovered from the culture supernatant. That is, the culture is treated by a method such as centrifugation to obtain the culture supernatant, and a purified preparation can be obtained from the culture supernatant by an isolation and purification method similar to the above.

The cations for gel formation are cations which bind to the anions for gel formation to form a gel. The cations for gel formation are typically multivalent cations and hydrogen ions (protons). The multivalent cation is a compound having a positive charge of divalent or more, and for example, a divalent or more metal cation and a compound having two or more cationic groups in one molecule are exemplified. As the compound having two or more cationic groups in one molecule, for example, an ion of polyamine having two or more amino groups in one molecule is exemplified. Examples of the divalent or more metal cation include alkaline-earth metal ions such as Ca²⁺ and Mg²⁺; transition metal ions such as Mn²⁺, Fe²⁺, Fe³⁺, Co²⁺, and Cu²⁺; Zn²⁺; and Al³⁺.

The anions for gel formation are anions which bind to the cations for gel formation to form a gel. Examples of the anions for gel formation include ions of polysaccharides having an anionic group such as carrageenan, pectin (for example, LM pectin or the like), gum arabic, xanthane gum, gellan gum, soybean polysaccharides, and alginic acid (for example, sodium alginate) and ions of acids such as polylactic acid and polyphosphoric acid.

The cations for gel formation and the anions for gel formation can be used arbitrarily in combination, but from the viewpoint of having excellent gel formation efficiency, it is preferable that the cations for gel formation are divalent or higher metal cations and the anions for gel formation are alginate ions, and it is more preferable that the cations for gel formation are Ca²⁺ and the anions for gel formation are alginate ions.

The polymer solution (dope) can be obtained, for example, by adding a polymer substance to a solvent having cations for gel formation dissolved therein, and dissolving the polymer substance by a known method such as stirring, heating, and electric field application.

The cations for gel formation may be added as a compound generating the cations for gel formation when being dissolved in a solvent (multivalent cation compound) to the solvent. Specific examples of the multivalent cation compound include metallic halide (for example, CaCl₂), MgCl₂), metallic nitrate (for example, Ca(NO₃)₂, Mg(NO₃)₂), metal thiocyanate (for example, Ca(SCN)₂, Mg(SCN)₂), a diamine compound (for example, ethylenediamine, p-phenylenediamine), and a multivalent complex salt (for example, [Co₄(OH)₆.en₆] (NO₃)₆).

The solvent of the polymer solution may be an aqueous solvent such as water and a buffer solution, and may be an organic solvent such as dimethylsulfoxide (DMSO), N,N-dimethylformamide (DMF), hexafluoroisopropanol (HFIP), and hexafluroacetone (HFAc). The solvent of the polymer solution may be a mixed solvent obtained by mixing two or more kinds of solvent. The mixed solvent also includes a mixed solvent of an aqueous solvent and an organic solvent.

The polymer solution may further contain a dissolution promoter which promotes dissolution of the polymer substance. According to this, the polymer solution is easily prepared. As a specific example of the dissolution promoter, for example, in a case where the polymer substance is a protein, for example, inorganic salts composed of Lewis acids and Lewis bases described below are exemplified. Examples of the Lewis bases include oxo-acid ions (such as nitrate ion and perchlorate ion), metal oxo-acid ions (such as permanganate ion), halide ions, thiocyanate ions, and cyanate ions. Examples of the Lewis acids include metal ions such as alkali metal ions and alkaline-earth metal ions, polyatomic ions such as ammonium ions, and complex ions. Specific examples of the inorganic salts composed of Lewis acids and Lewis bases include lithium salts such as lithium chloride, lithium bromide, lithium iodide, lithium nitrate, lithium perchlorate, and lithium thiocyanate, calcium salts such as calcium chloride, calcium bromide, calcium iodide, calcium nitrate, calcium perchlorate, and calcium thiocyanate, iron salts such as iron chloride, iron bromide, iron iodide, iron nitrate, iron perchlorate, and iron thiocyanate, aluminum salts such as aluminum chloride, aluminum bromide, aluminum iodide, aluminum nitrate, aluminum perchlorate, and aluminum thiocyanate, potassium salts such as potassium chloride, potassium bromide, potassium iodide, potassium nitrate, potassium perchlorate, and potassium thiocyanate, sodium salts such as sodium chloride, sodium bromide, sodium iodide, sodium nitrate, sodium perchlorate, and sodium thiocyanate, zinc salts such as zinc chloride, zinc bromide, zinc iodide, zinc nitrate, zinc perchlorate, and zinc thiocyanate, magnesium salts such as magnesium chloride, magnesium bromide, magnesium iodide, magnesium nitrate, magnesium perchlorate, and magnesium thiocyanate, barium salts such as barium chloride, barium bromide, barium iodide, barium nitrate, barium perchlorate, and barium thiocyanate, and strontium salts such as strontium chloride, strontium bromide, strontium iodide, strontium nitrate, strontium perchlorate, and strontium thiocyanate. In a case where the polymer substance is vulcanized rubber, specific examples of the dissolution promoter include thiol and amine. In a case where the polymer substance is cellulose, specific examples of the dissolution promoter include carbon disulfide and sodium hydroxide. In a case where the polymer substance is a synthetic resin, for example, an acrylic resin, specific examples of the dissolution promoter include sodium thiocyanate, in a case where the polymer substance is polyamide and polyparaphenylene terephthalamide, specific examples of the dissolution promoter include sulfuric acid, and in a case where the polymer substance is polyvinyl chloride, specific examples of the dissolution promoter include acetone and cyclohexane. Incidentally, depending on a combination of the polymer substance and the cations for gel formation, the cations for gel formation may act as a dissolution promoter of promoting dissolution of the polymer substance.

The content of the cations for gel formation in the polymer solution is appropriately set according to types and the like of the polymer substance, the cations for gel formation, the anions for gel formation, the solvent, and other additives. For example, in a case where the polymer substance is a protein, the cations for gel formation are Ca²⁺, and the anions for gel formation are alginate ions, the content of the cations for gel formation is preferably in a concentration range of 0.1 to 5.0 mol/L and more preferably in a concentration range of 0.4 to 3.0 mol/L.

The content of the polymer substance in the polymer solution is appropriately set according to the type of the polymer substance, and the like. For example, in a case where the polymer substance is a protein, the content of the polymer substance is preferably 10 to 50 mass % and more preferably 15 to 30 mass % based on the total amount of the polymer solution.

The viscosity of the polymer solution is not particularly limited, but for example, from the viewpoint of facilitating molding, the viscosity is preferably 50 to 20000 cP and more preferably 200 to 8000 cP at 25° C. The viscosity of the polymer solution is measured, for example, by an EMS viscometer.

(Anion Solution)

The anion solution (coagulation liquid) can be obtained, for example, by adding the anions for gel formation to a solvent, and dissolving the anions for gel formation by a known method such as stirring, heating, and electric field application.

The anions for gel formation may be added as a compound generating the anions for gel formation when being dissolved in a solvent (anion compound) to the solvent. Specific examples of the anion compound include polysaccharides having an anionic group and salts thereof. Preferred specific examples of the polysaccharides having an anionic group and salts thereof include sodium alginate. The sodium alginate is represented by (NaC₆H₇O₆). (n is 1 to 5000), and is abbreviated as Na-ALG in some cases.

The solvent of the anion solution may be an aqueous solvent such as water and a buffer solution, and may be an organic solvent such as dimethylsulfoxide (DMSO), N,N-dimethylformamide (DMF), hexafluoroisopropanol (HFIP), and hexafluroacetone (HFAc). The solvent of the anion solution may be a mixed solvent obtained by mixing two or more kinds of solvent. The mixed solvent also includes a mixed solvent of an aqueous solvent and an organic solvent.

The solvent of the anion solution may be the same as the solvent of the polymer solution. In addition, both the solvents may be an aqueous solvent. When both the polymer solution (dope) and the anion solution (coagulation liquid) are set to an aqueous solvent, the polymer aggregate can be formed in a water system. According to this, the polymer aggregate thus obtained is applicable to a living body, and for example, can be used instead of silken threads currently used as surgical sewing threads. Moreover, the polymer aggregate is applicable to artificial blood vessels, artificial skins, artificial bones, and the like.

The anion solution may further contain an aggregation promoter for the polymer substance. According to this, the production efficiency of the polymer aggregate can be improved. As a specific example of the aggregation promoter for the polymer substance, for example, in a case where the polymer substance is a protein, alkali metal ions such as Nat, Lit, and K⁺ are exemplified. In a case where the polymer substance is cellulose, as a specific example of the aggregation promoter for the polymer substance, ethanol is exemplified. In a case where the polymer substance is non-vulcanized rubber, as a specific example of the aggregation promoter for the polymer, a disulfide compound is exemplified.

The content of the anions for gel formation in the anion solution is appropriately set according to types and the like of the polymer substance, the cations for gel formation, the anions for gel formation, the solvent, and other additives. For example, in a case where the polymer substance is a protein, the cations for gel formation are Ca²⁺, and the anions for gel formation are alginate ions, the content of the anions for gel formation is preferably in a concentration range of 0.1 to 6.0 weight % and more preferably in a concentration range of 1.0 to 2.0 weight %.

(Gel Formation Step)

In the gel formation step, a polymer solution is brought into contact with an anion solution to form a gel at an interface between the polymer solution and the anion solution. When the polymer solution is brought into contact with the anion solution, by controlling the form of a contact surface, a gel in an arbitrary form such as a fibrous form, a film form, a hollow pipe form, and a bead form can be formed.

In the case of a fibrous form, for example, a gel can be molded by the polymer solution being continuously extruded from a spinning nozzle to the anion solution. In the case of a film form, for example, a gel can be molded by the polymer solution being continuously extruded from a slit hole to the anion solution. In the case of a hollow pipe form, for example, a gel can be molded by the polymer solution being continuously extruded from a hollow slit hole or through a double tube to the anion solution and the polymer solution also being injected to the inside of the polymer solution. In the case of a bead form, for example, a gel can be molded by the polymer solution being intermittently extruded from a nozzle to the anion solution.

(Aggregation Step)

In the aggregation step, the polymer substance is aggregated inside the gel. In the aggregation step, the gel functions as a dialysis membrane, and for example, excessive cations for gel formation or dissolution promoter contained in the polymer solution is discharged through the gel into the anion solution so that aggregation of the polymer substance proceeds. Further, for example, the aggregation promoter contained in the anion solution infiltrates through the gel into the polymer solution so that aggregation of the polymer substance proceeds. In the aggregation step, for example, the anion solution may be stirred. According to this, the efficiency of substance transfer through the gel is improved, and thus production efficiency is improved.

The time for executing the aggregation step may be appropriately set according to types and the like of the polymer substance, the cations for gel formation, the anions for gel formation, the solvent, and other additives and the form, application, and the like of the polymer aggregate, but is typically 3 minutes to 3 hours and preferably 3 minutes to 2 hours.

By executing the aggregation step, a polymer aggregate to which the gel is bound in a sheath form is obtainable.

(Aggregation Step 2)

The production method according to this embodiment may further include a step for immersing the polymer aggregate to which the gel is bound in a sheath form in a solvent such as water (also referred to as “aggregation step 2”) after the aggregation step. According to this, movement of the cations for gel formation or the dissolution promoter from the inside of the gel to the solvent is promoted so that the polymer aggregate can be further aggregated.

The time for executing the aggregation step 2 may be appropriately set according to types and the like of the polymer substance, the cations for gel formation, the anions for gel formation, the solvent, and other additives and the form, application, and the like of the polymer aggregate, but is typically 3 minutes to 3 hours, preferably 3 minutes to 2 hours, and more preferably 3 minutes to 1 hour.

(Gel Removal Step)

The production method according to this embodiment may further include a step for removing the gel from the polymer aggregate (also referred to as “gel removal step”) after the aggregation step or the aggregation step 2. As for the polymer aggregate, the polymer aggregate to which the gel is bound in a sheath form may be provided without change according to the application, and in a case where the gel is unnecessary, the polymer aggregate from which the gel is removed by the gel removal step may be provided. As the method of removing the gel, for example, a removal method using a mechanical means such as crushing the gel using a roller, a chemical method of immersing the gel in a solution that does not dissolve the polymer aggregate but dissolves only the gel, or a method of removing the gel by ultrasonic wave or heat 15 exemplified.

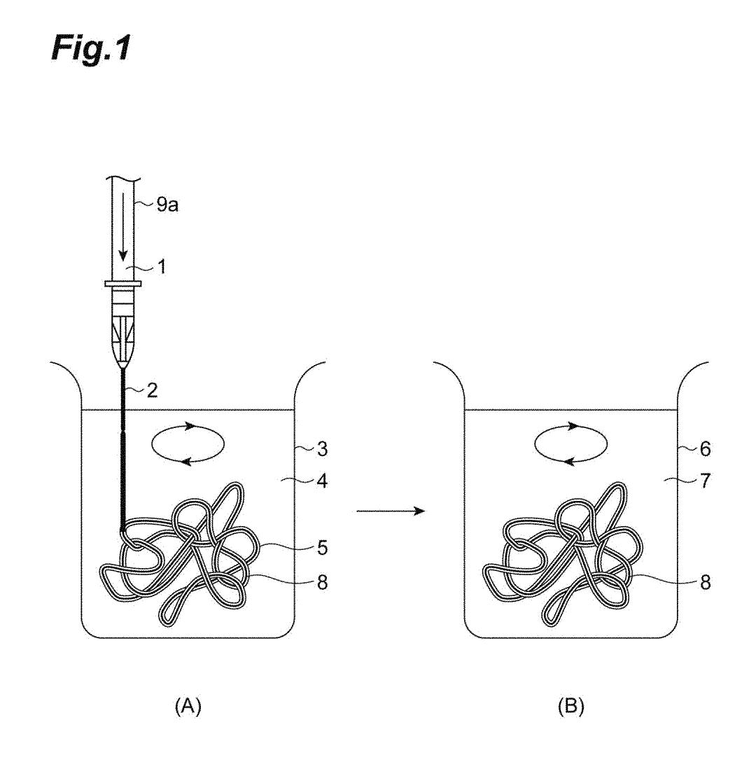

The production method according to this embodiment will be further described with reference to FIGS. 1 to 6.

FIG. 1 is an explanatory diagram illustrating a production method according to an embodiment. In the production method illustrated in FIG. 1, a polymer aggregate in a fibrous form is obtained. FIG. 1, drawing (A) is an explanatory diagram illustrating a step for extruding a polymer solution (dope) into an anion solution (coagulation liquid). A dope 1 is put in a syringe 9a, and the dope 1 is continuously extruded from a nozzle to a coagulation liquid 4. The dope 1 is obtained by a polymer substance (for example, a polypeptide derived from a spider silk protein) being dissolved in an aqueous solution containing cations for gel formation (for example, Ca′). The coagulation liquid 4 is an aqueous solution containing anions for gel formation (for example, alginate ions). The coagulation liquid 4 has been put in a container 3 and slowly stirred. An extruded dope 2 forms a gel 5 at an interface in the coagulation liquid 4. The polymer substance (for example, a polypeptide derived from a spider silk protein) aggregates inside the gel 5 to form a polymer aggregate 8. Since the gel 5 in a fibrous form is formed by extrusion, the polymer aggregate 8 thus obtained also has a fibrous form.

FIG. 1, drawing (B) is an explanatory diagram illustrating a step for immersing a polymer aggregate covered with a gel in water and further causing the polymer aggregate to aggregate. Water 7 has been put in a container 6 and slowly stirred. The polymer aggregate 8 covered with the gel 5 is immersed in the water 7 with the gel 5. According to this, the polymer aggregate 8 further aggregates. This polymer aggregate is dried to thereby obtain threads with high strength.

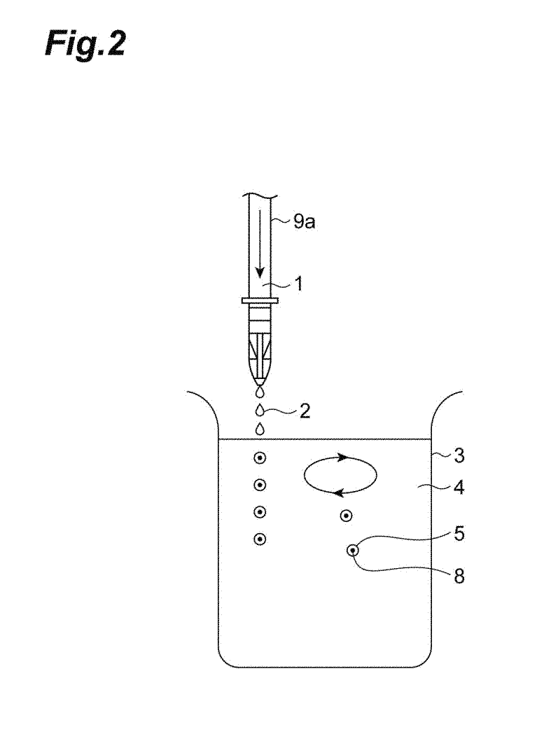

FIG. 2 is an explanatory diagram illustrating a production method according to another embodiment. In the production method illustrated in FIG. 2, a polymer aggregate in a particulate form (bead form) is obtained. The dope 1 is put in the syringe 9a, and the dope 1 is intermittently extruded from the nozzle to the coagulation liquid 4.

The extruded dope 2 forms the gel 5 at the interface in the coagulation liquid 4. The polymer substance aggregates inside the gel 5 to form the polymer aggregate 8. Since the gel 5 in a particulate form is formed by intermittent extrusion, the polymer aggregate 8 thus obtained also has a particulate form.

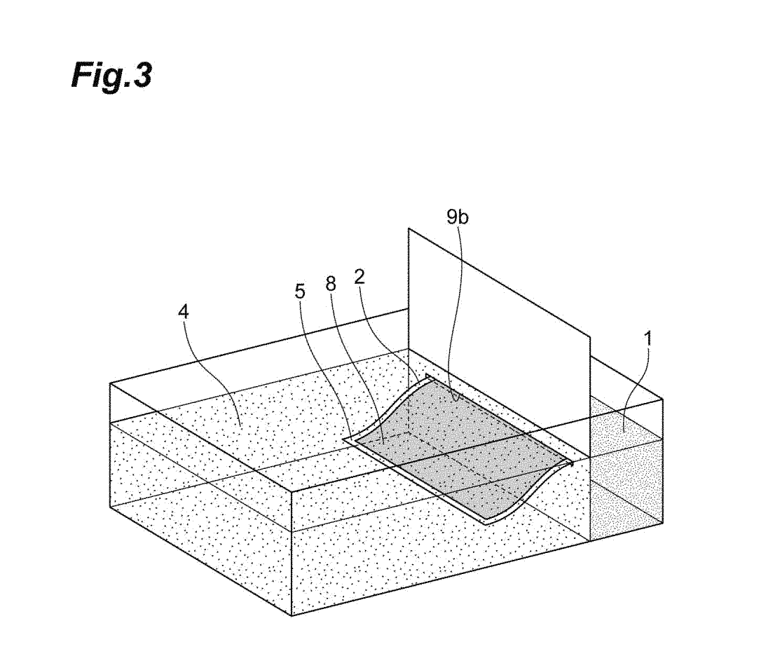

FIG. 3 is an explanatory diagram illustrating a production method according to another embodiment. In the production method illustrated in FIG. 3, a polymer aggregate in a film form is obtained. The dope 1 is continuously extruded from a slit hole 9b to the coagulation liquid 4. The extruded dope 2 forms the gel 5 in the coagulation liquid 4. The polymer substance aggregates inside the gel 5 to form the polymer aggregate 8. Since the gel 5 in a film form is formed by extrusion, the polymer aggregate 8 thus obtained also has a film form.

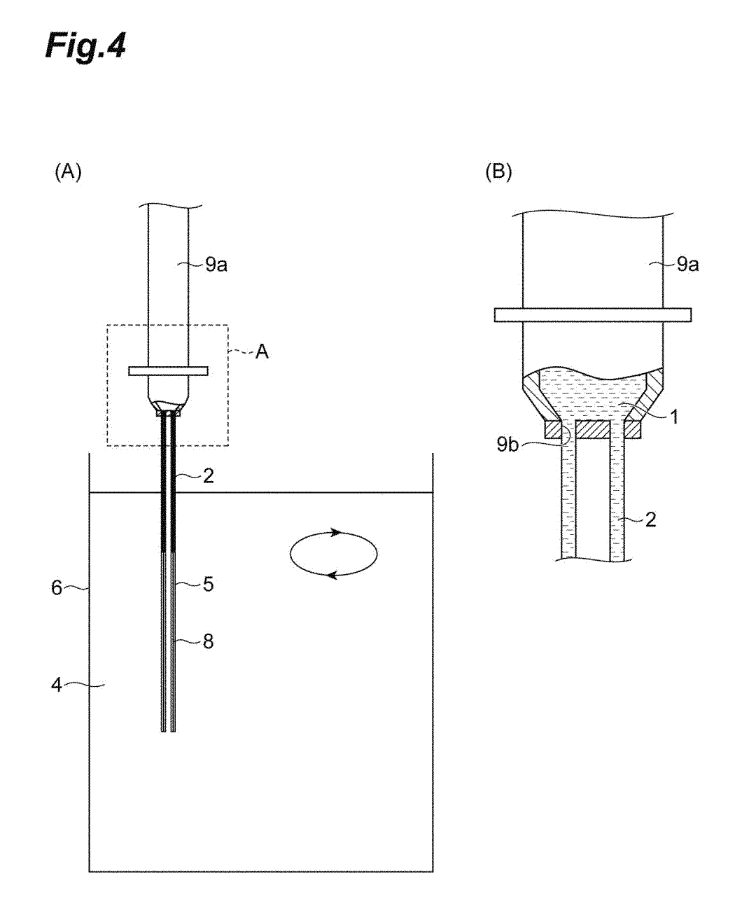

FIG. 4, drawing (A) is an explanatory diagram illustrating a production method according to another embodiment. FIG. 4, drawing (B) is an enlarged view of a part surrounded by A in FIG. 4, drawing (A). In the production method illustrated in FIG. 4, drawing (A), a polymer aggregate in a hollow pipe form or tubular form is obtained. The dope 1 is continuously extruded to the coagulation liquid 4 from the syringe 9a through a circular slit hole 9b, which is provided at a tip of the nozzle, to have a tubular form. The extruded dope 2 forms the gel 5 in a tubular form in the coagulation liquid 4. The polymer substance aggregates inside the gel 5 to form the polymer aggregate 8. Since the gel 5 in a tubular form is formed by extrusion, the polymer aggregate 8 thus obtained also has a small-diameter hollow pipe form or tubular form.

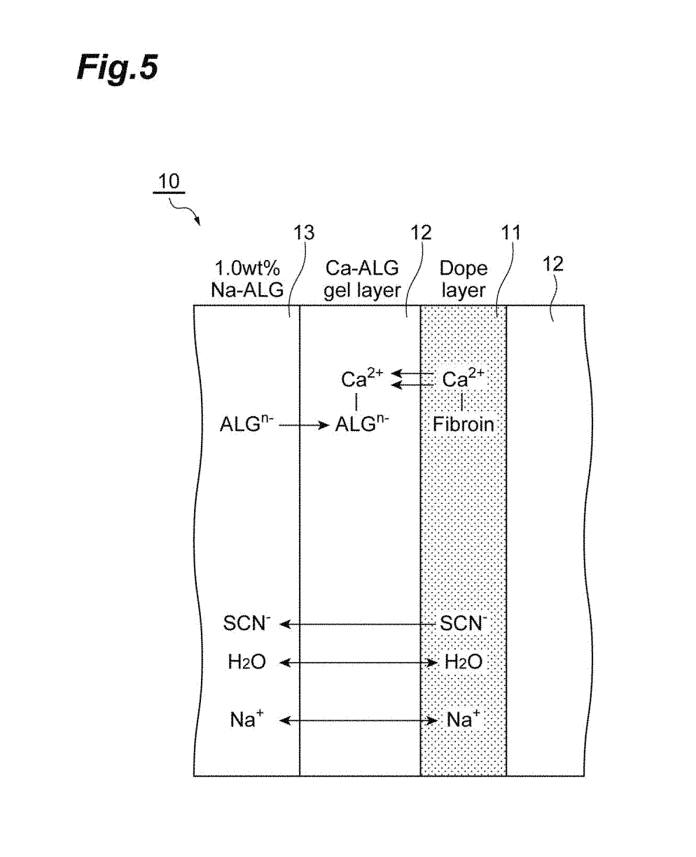

FIG. 5 is a schematic cross-sectional view illustrating a coagulation liquid-gel-dope interface in a production method according to an embodiment. The mechanism in which a polymer aggregate is formed by the production method according to the present invention will be described using a schematic cross-sectional view 10 illustrating the coagulation liquid-gel-dope interface illustrated in FIG. 5. In FIG. 5, a polymer solution containing Ca(SCN)₂ and fibroin is used as the dope, and an anion solution containing sodium alginate is used as the coagulation liquid. A gel layer 12 is formed from Ca²⁺ (cations for gel formation) contained in the dope and alginate ions (ALGⁿ: anions for gel formation) contained in the coagulation liquid. That is, ion-exchange reaction occurs between Ca²⁺-fibroin of a dope layer 11 and sodium alginate (NatALGⁿ) of a coagulation liquid layer 13 to generate Ca²⁺-ALGⁿ, thereby obtaining a gel. Thus, the gel layer 12 is formed. The gel layer 12 is considered to function as a dialysis membrane. As a result, low-molecular weight SCN⁻, H₂O, and Na⁺ move through the gel layer 12, whereas the fibroin that is a high molecule remains inside the gel. Further, it is considered that solubility of the fibroin in the inside of the gel is degraded by outflow of SCN⁻ through the gel layer 12, and thus aggregation proceeds.

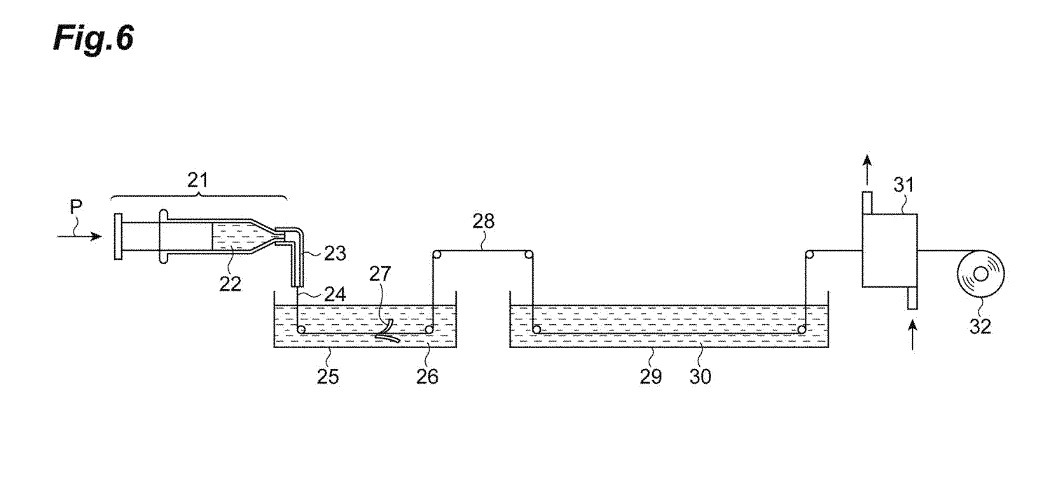

FIG. 6 is a production process diagram according to an embodiment. A dope 22 is extruded from a nozzle 23 of an extruder 21 in a P direction, an extruded dope 24 is immersed in a coagulation liquid 26 of a coagulation bath 25, a gel 27 thus obtained is peeled off, an aggregated fibroin 28 is further aggregated by being immersed in pure water 30 of a water bath 29, and then the aggregated fibroin is caused to pass through a dryer 31 to be solidified, thereby obtaining a yarn roll 32. In this way, fibroin threads can be produced by a series of processes. The fibroin threads has practically sufficient strength even in a state of unstretched threads, but may be further stretched.

### EXAMPLES

Hereinafter, the present invention will be described in more detail based on Examples. However, the present invention is not limited to the following Examples.

### Example 1

1. Preparation of Polypeptide Derived from Spider Silk Protein

### <Gene Synthesis>

### (1) Gene Synthesis of ADF3Kai

A partial amino acid sequence of ADF3 (GI No.: 1263287), which is one of two principal dragline silk proteins of Araneus diadematus, was obtained from NCBI web database, and synthesis of a gene encoding an amino acid sequence (SEQ ID NO: 2), which is obtained by adding an amino acid sequence (SEQ ID NO: 4) composed of a start codon, His 10-tag and HRV3C Protease (Human rhinovirus 3C Protease) recognition site to the N-terminal of the partial amino acid sequence of ADF3, was outsourced to GenScript, Inc. As a result, a pUC57 vector to which a gene of ADF3Kai having a base sequence represented by SEQ ID NO: 5 had been introduced was obtained (having an Nde I site immediately upstream of 5′ terminal of the gene and an Xba I site immediately downstream of 5′ terminal thereof). Thereafter, the gene was subjected to a restriction enzyme treatment with Nde I and EcoR I, and recombined into a pET22b(+) expression vector.

### (2) Gene Synthesis of ADF3Kai-Large

The half of the gene sequence of ADF3Kai on the 5′ side (hereinafter, referred to as a sequence A) was amplified by the PCR reaction using ADF3Kai as a template, and a T7 promoter primer (SEQ ID NO: 14) and a Rep Xba I primer (SEQ ID NO: 15). The obtained DNA fragment of the sequence A was recombined into a pUC118 vector that had been subjected to the restriction enzyme treatment with Nde I and Xba I in advance using a Mighty Cloning Kit (manufactured by Takara Bio Inc.). Similarly, the half of the gene sequence of ADF3Kai on the 3′ side (hereinafter, referred to as a sequence B) was amplified by the PCR reaction using ADF3Kai as a template, and an Xba I Rep primer (SEQ ID NO: 16) and a T7 terminator primer (SEQ ID NO: 17). The obtained DNA fragment of the sequence B was recombined into a pUC118 vector that had been subjected to the restriction enzyme treatment with Xba I and EcoR I in advance using the Mighty Cloning Kit (manufactured by Takara Bio Inc.). The pUC118 vector to which the sequence A had been introduced and the pUC118 vector to which the sequence B had been introduced were subjected to the restriction enzyme treatment with Xba I and EcoR I, respectively, and target DNA fragments of the sequences A and B were purified by gel cut. The DNA fragments A and B and the pET22b(+) that had been subjected to the restriction enzyme treatment with Nde I and EcoR I in advance were subjected to a ligation reaction and transformed into Escherichia coli DH5α. After confirmation of the insertion of the target DNA fragments by a colony PCR using a T7 promoter primer and a T7 terminator primer, plasmid was extracted from a colony where a target band size (3.6 kbp) was obtained, and the entire base sequence was checked by a sequence reaction using a 3130xl Genetic Analyzer (Applied Biosystems). Consequently, the construction of a gene of ADF3Kai-Large represented by SEQ ID NO: 6 was confirmed. The amino acid sequence of ADF3Kai-Large is as represented by SEQ ID NO: 3.

### (3) Gene Synthesis of ADF3Kai-Large-NRSH1

With a pET22b(+) vector to which the gene of ADF3Kai-Large obtained above had been introduced used as a template, through site-directed mutagenesis using a PrimeStar Mutagenesis Basal Kit (manufactured by Takara Bio Inc.), a codon GGC corresponding to the 1155th amino acid residue, i.e., glycine (Gly), in the amino acid sequence of ADF3Kai-Large (SEQ ID NO: 3) was mutated into a stop codon TAA, and a gene of ADF3Kai-Large-NRSH1 represented by SEQ ID NO: 7 was constructed on the pET22b(+). The accuracy of the introduction of the mutation was checked by the sequence reaction using the 3130xl Genetic Analyzer (Applied Biosystems). Incidentally, the amino acid sequence of ADF3Kai-Large-NRSH1 (hereinafter, also simply referred to as “NRSH1”) is as represented by SEQ ID NO: 1.

### <Expression of Protein>

The pET22b(+) expression vector containing the gene sequence of NRSH1 obtained above was transformed into Escherichia coli Rosetta (DE3). The obtained single colony was cultured for 15 hours in 2 mL of an LB culture medium containing ampicillin. Thereafter, 1.4 mL of the culture solution was added to 140 mL of an LB culture medium containing ampicillin, and cultured to an OD₆₀₀ of 3.5 under the conditions of 37° C. and 200 rpm. Next, the culture solution with the OD₆₀₀ of 3.5 was added to 7 L of a 2×YT culture medium containing ampicillin together with 140 mL of 50% glucose, and further cultured to the OD₆₀₀ of 4.0. Thereafter, isopropyl-β-thiogalactopyranoside (IPTG) was added to the obtained culture solution with the OD₆₀₀ of 4.0 such that the final concentration would be 0.5 mM, thereby inducing the expression of protein. After a lapse of two hours from the addition of IPTG the culture solution was centrifuged and bacterial cells were collected. Protein solutions prepared from the culture solution before the addition of IPTG and after the addition of IPTG were each electrophoresed in a polyacrylamide gel. Consequently, a band having a target size (about 101.1 kDa) was observed with the addition of IPTG, and the expression of the target protein was confirmed.

### <Preparation of Polypeptide>

(I) About 4.5 g of bacterial cells of Escherichia coli expressing the NRSH1 protein and 30 mL of a buffer solution AI (20 mM Tris-HCl, pH 7.4) were added to a centrifuge tube (50 mL). After dispersing the bacterial cells with a mixer (“SI-0286” manufactured by GE, level 10), the dispersion was centrifuged (10,000 rpm, 10 minutes, room temperature) with a centrifuge (“MX-305” manufactured by TOMY SEIKO CO., LTD.), and a supernatant was discarded.

(II) To a precipitate (bacterial cells) obtained by the centrifugation, 30 mL of the buffer solution AI and 0.3 mL of 0.1 M PMSF (dissolved by isopropanol) were added, and the precipitate was dispersed for 3 minutes with the mixer (level 10) manufactured by GE. Thereafter, the bacterial cells were disrupted using a sonicator (“VCX 500” manufactured by Sonics & Materials, Inc.) and centrifuged (10,000 rpm, 10 minutes, room temperature).

(III) To the precipitate obtained by the centrifugation, 30 mL of the buffer solution AI was added, and the precipitate was dispersed for 3 minutes with a mixer (“T 18 basic ULTRA-TURRAX” manufactured by IKA, level 2). Thereafter, the dispersion was centrifuged (10,000 rpm, 10 minutes, room temperature) with the centrifuge manufactured by TOMY SEIKO CO., LTD., and a supernatant was removed.

(IV) To the centrifuge tube from which a supernatant is discarded, 7.5 M of a urea buffer solution I (7.5 M urea, 10 mM sodium dihydrogen phosphate, 20 mM NaCl, 1 mM Tris-HCl, pH 7.0) was added, the precipitate was dispersed well with the sonicator (“VCX 500” manufactured by Sonics & Materials, Inc.) (level 7). Thereafter, the dispersion was dissolved for 120 minutes (200 rpm, 60° C.) with a shaker (“BR-43FL/MR” manufactured by TAITEC CORPORATION). The protein solution after being dissolved was centrifuged (11,000×g, 10 minutes, room temperature) with the centrifuge manufactured by TOMY SEIKO CO., LTD., a supernatant was dialyzed with water using a dialysis tube (cellulose tube 36/32 manufactured by Sanko Junyaku Co., Ltd.). The white aggregated protein obtained after the dialysis was collected by centrifugation, and moisture was removed by a freeze dryer to collect freeze-dried powder. The purification degree of the target protein NRSH1 (about 101.1 kDa) in the obtained freeze-dried powder was checked by analyzing images of the results of polyacrylamide gel electrophoresis (CBB staining) of the protein powder using Totallab (nonlinear dynamics ltd.). As a result, the purification degree of NRSH1 was about 85%.

2. Preparation of Polymer Solution (Dope)

The spider silk protein (NRSH1) was added to a pure water solution of 1 M Ca(SCN)₂ to have a concentration of 20 mass %, and dissolved for 3 hours using a shaker. Thereafter, dusts and bubbles were removed to obtain a dope. The solution viscosity of the dope was 2088 cP (centipoises) at 25° C.

3. Preparation of Anion Solution (Coagulation Liquid)

Pure water containing 1.0 mass % of sodium alginate (Na-ALG) was used as a coagulation liquid.

4. Production of Polymer Aggregate

As illustrated in FIG. 1, drawing (A), a dope was extruded using a syringe to a coagulation liquid. The diameter of an extruding nozzle was set to 0.5 mm and the extruding speed was set to 50 mL/Hr. After extruding, the dope was immersed for 90 minutes in the coagulation liquid (gelling bath immersion time) to obtain a polymer aggregate formed in the gel. Thereafter, the gel covering the polymer aggregate was removed to obtain filamentous fibroin.

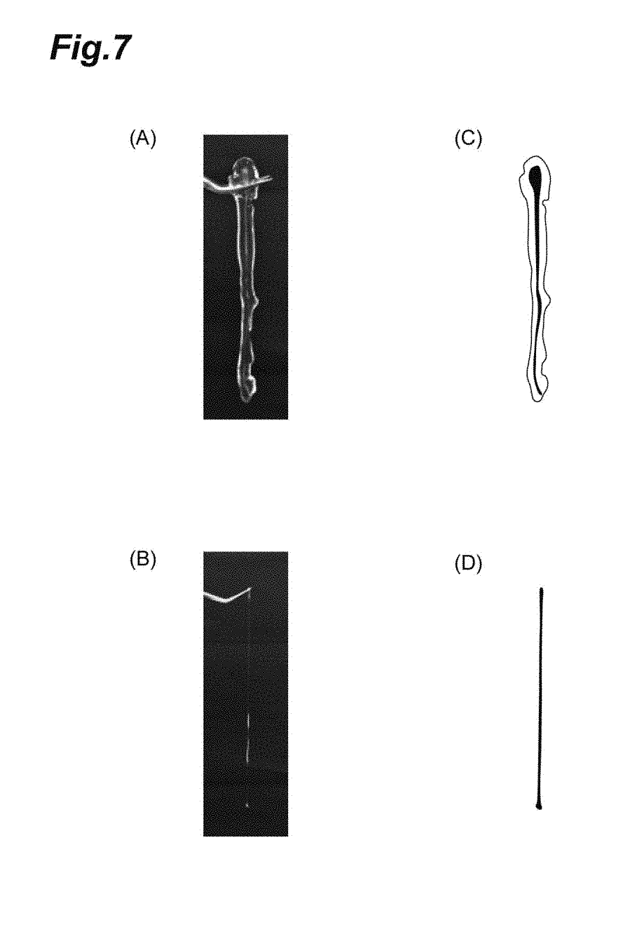

The production conditions and results are presented in Table 1 and FIG. 7. FIG. 7 is a photograph showing a polymer aggregate obtained in Example 1 (in which spider silk proteins aggregate to be formed in a fiber form) and a trace drawing which traces the photograph. FIG. 7, drawing (A) is a photograph showing a polymer aggregate formed in a gel. FIG. 7, drawing (B) is a photograph showing the polymer aggregate from which the gel is removed. FIG. 7, drawing (C) is a trace drawing which traces the photograph of FIG. 7, drawing (A). FIG. 7, drawing (D) is a trace drawing which traces the photograph of FIG. 7, drawing (B).

### Example 2

A polymer aggregate was produced in a similar manner to Example 1, except that a dope was prepared by adding the spider silk protein (NRSH1) to a DMSO solution of 1 M CaCl₂) such that the concentration would be 20 mass % and the gelling bath immersion time was changed to 30 minutes.

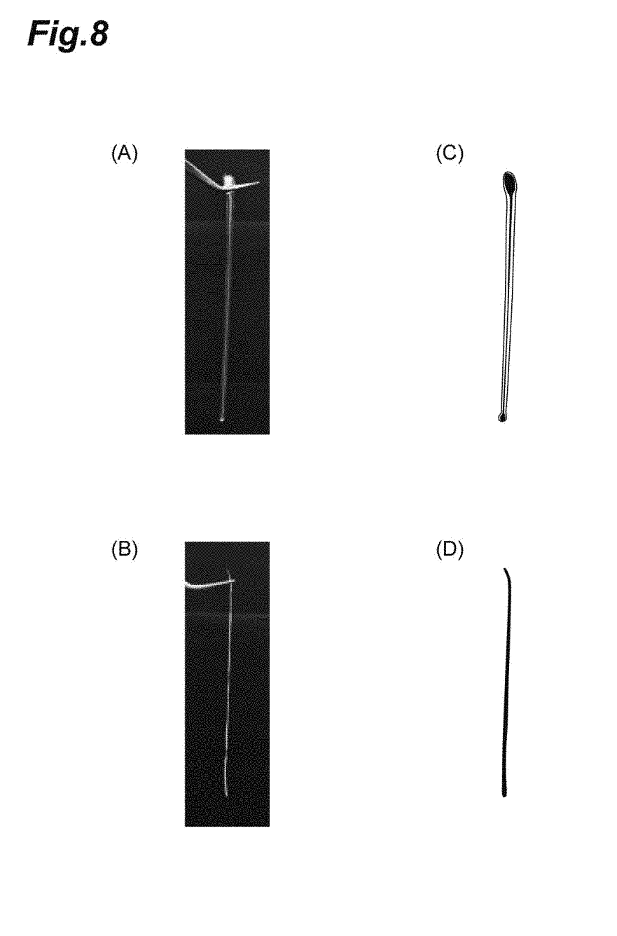

The production conditions and results are presented in Table 1 and FIG. 8. FIG. 8 is a photograph showing a polymer aggregate obtained in Example 2 (in which spider silk proteins aggregate to be formed in a fiber form) and a trace drawing which traces the photograph. FIG. 8, drawing (A) is a photograph showing a polymer aggregate formed in a gel. FIG. 8, drawing (B) is a photograph showing the polymer aggregate from which the gel is removed. FIG. 8, drawing (C) is a trace drawing which traces the photograph of FIG. 8, drawing A). FIG. 8, drawing (D) is a trace drawing which traces the photograph of FIG. 8, drawing (B).

### Example 3

A polymer aggregate was produced in a similar manner to Example 1, except that silken thread (not removing sericin) was used instead of the spider silk protein (NRSH1), the concentration of Ca(SCN)₂ contained in the dope was adjusted to 5 M, the gelling bath immersion time was changed to 180 minutes, and the silk was immersed for 60 minutes in RO water (RO water immersion time) after being immersed in the coagulation liquid.

As the silken thread (not removing sericin), silken thread obtained by taking a silkworm pupa out from the cocoon and cutting the cocoon shell was used.

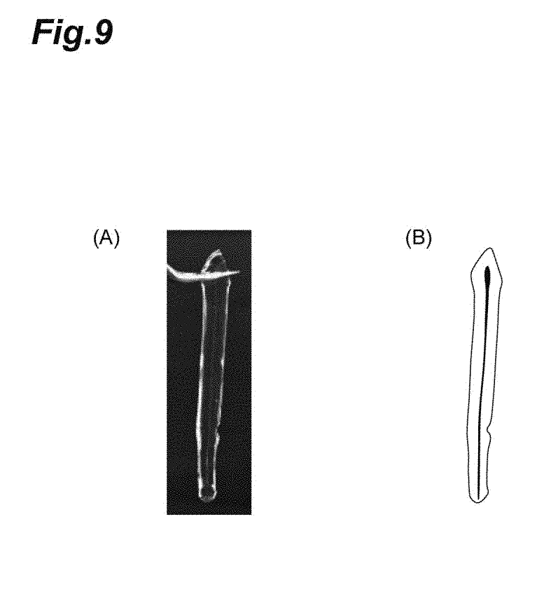

The production conditions and results are presented in Table 1 and FIG. 9. FIG. 9 is a photograph showing a polymer aggregate obtained in Example 3 (in which silk proteins aggregate to be formed in a fiber form) and a trace drawing which traces the photograph. FIG. 9, drawing (A) is a photograph showing a polymer aggregate formed in a gel. FIG. 9, drawing (B) is a trace drawing which traces the photograph of FIG. 9, drawing (A). Incidentally, the polymer aggregate of Example 3 was integrated with the gel.

### Example 4

A polymer aggregate was produced in a similar manner to Example 3, except that filamentous fibroin obtained by removing sericin from the silken thread was used instead of the silken thread (not removing sericin), the sodium alginate concentration in the coagulation liquid was changed to 2.0 mass %, the gelling bath immersion time was changed to 30 minutes, and the RO water immersion time was changed to 30 minutes.

The filamentous fibroin obtained by removing sericin from the silken thread was prepared as follows.

(1) Silken thread was put into 0.5 mass % of boiling Marseilles soap water (Marseilles soap was grated by a grater for use) such that the bath ratio would be 1:100, and was heated for 30 minutes while stirring occasionally.

(2) The soap water was discarded and the silken thread was rinsed with lukewarm water once.

(3) The silken thread rinsed with lukewarm water in (2) was put in boiling water and heated such that the bath ratio would be 1:100.

(4) The hot water was discarded.

(5) The procedures (1) to (4) were repeated two more times (three times in total).

(6) Finally, the silken thread was dehydrated and dried at 60° C. or lower.

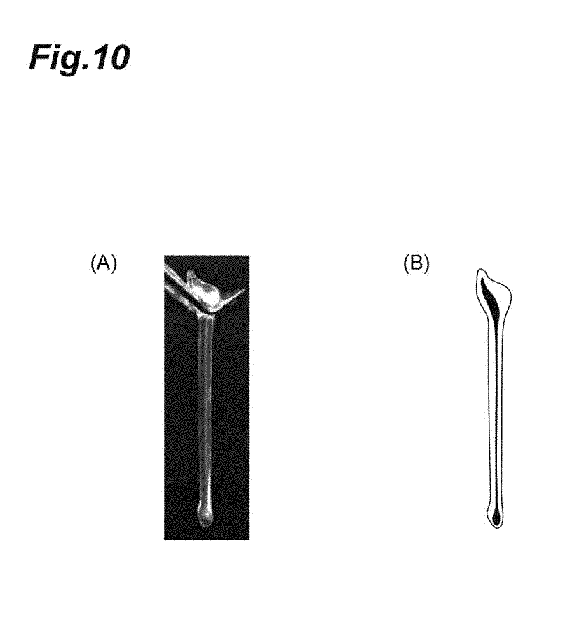

The production conditions and results are presented in Table 1 and FIG. 10. FIG. 10 is a photograph showing a polymer aggregate obtained in Example 4 (in which silk proteins aggregate to be formed in a fiber form) and a trace drawing which traces the photograph. FIG. 10, drawing (A) is a photograph showing a polymer aggregate formed in a gel. FIG. 10, drawing (B) is a trace drawing which traces the photograph of FIG. 10, drawing (A). Incidentally, the polymer aggregate of Example 4 was integrated with the gel.

### Example 5

A polymer aggregate was produced in a similar manner to Example 4, except that regenerated silken thread was used instead of the filamentous fibroin obtained by removing sericin from the silken thread.

The regenerated silken thread was prepared as follows.

(1) Silken thread was heated for 30 minutes in 0.5 mass % of boiling Marseilles soap water (Marseilles soap was grated by a grater for use).

(2) Thereafter, the silken thread was heated for 30 minutes in boiling water.

(3) The procedures (1) and (2) were repeated two more times (three times in total).

(4) Finally, the silken thread was heated for 30 minutes in boiling water.

(5) The silken thread was dried overnight in an environment of a temperature of 37° C.

(6) The silk after drying was weighed, an aqueous solution of 9 M lithium bromide was added thereto in an amount of 10 times the weight of the silk, and the silk was dissolved in an environment of 40° C.

(7) The dissolved solution obtained in (6) was placed in a cellulose dialysis membrane (Seamless Cellulose Tubing manufactured by VISKASESELES COAP, 36/32), and dialyzed with pure water for 3 to 4 days.

(8) The collected solution after dialysis was filtered using filters (hole diameters of 197 μm and 560 μm) to remove dusts.

(9) The protein concentration was measured by a BCA method, and the solution was diluted with MilliQ such that the protein concentration would be 2 mass % or less.

(10) The regenerated silken thread solution was freeze-dried in an environment of −80° C. The thickness was 1 to 2 cm.

(11) After freeze-drying, the solution was stored at 4° C.

The production conditions and results are presented in Table 1. Incidentally, the polymer aggregate of Example 5 was integrated with the gel.

### Example 6

A polymer aggregate was produced in a similar manner to Example 1, except that a commercially available gelatin (Catalog Number 170-6537 manufactured by Bio-Rad Laboratories) was used instead of the spider silk protein (NRSH1) and the gelling bath immersion time was changed to 30 minutes.

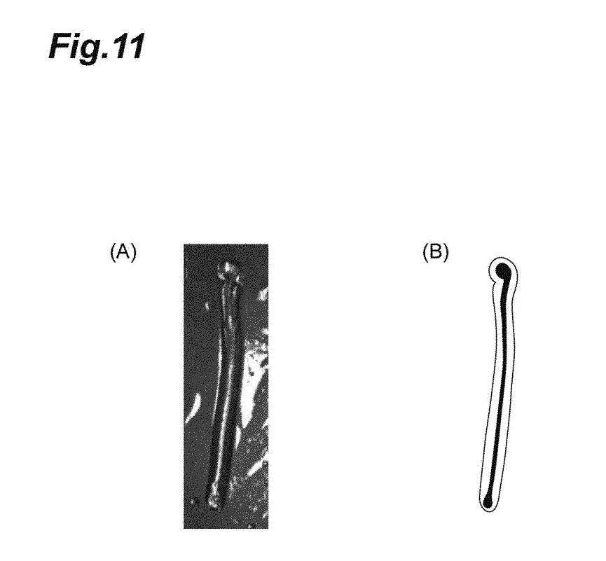

The production conditions and results are presented in Table 1 and FIG. 11. FIG. 11 is a photograph showing a polymer aggregate obtained in Example 6 (in which gelatin proteins aggregate to be formed in a fiber form) and a trace drawing which traces the photograph. FIG. 11, drawing (A) is a photograph showing a polymer aggregate formed in a gel. FIG. 11, drawing (B) is a trace drawing which traces the photograph of FIG. 11, drawing (A). Incidentally, the polymer aggregate of Example 6 was integrated with the gel.

TABLE 1

| | Example 1 | Example 2 | Example 3 | Example 4 | Example 5 | Example 6 |
|---|---|---|---|---|---|---|
| Type of protein | Spider | Spider | Silken | Silken | Regenerated | Gelatin |
| | silk | silk | thread | thread | silken | |
| | (NRSH1) | (NRSH1) | containing | removing | thread | |
| | | | sericin | sericin | | |
| Protein concentration | 20 | 20 | 20 | 20 | 20 | 20 |
| (mass %) | | | | | | |
| Solvent of protein | H₂O | DMSO | H₂O | H₂O | H₂O | H₂O |
| solution | | | | | | |
| Salt contained in dope | Ca(SCN)₂ | CaCl₂ | Ca(SCN)₂ | Ca(SCN)₂ | Ca(SCN)₂ | Ca(SCN)₂ |
| Concentration of salt | 1M | 1M | 5M | 5M | 5M | 1M |
| contained in dope | | | | | | |
| Salt contained in | Na-ALG | Na-ALG | Na-ALG | Na-ALG | Na-ALG | Na-ALG |
| coagulation liquid | | | | | | |
| Concentration of salt | 1.0 | 1.0 | 1.0 | 2.0 | 2.0 | 1.0 |
| contained in coagulation | | | | | | |
| liquid (mass %) | | | | | | |
| Gelling bath immersion | 90 | 30 | 180 | 30 | 30 | 30 |
| time (min) | | | | | | |
| RO water immersion | — | — | 60 | 30 | 30 | — |
| time (min) | | | | | | |
| Color of polymer | White | White | White | White | White | Transparent |
| aggregate | | | | | | |
| State of polymer | Easily | Easily | Integrated | Integrated | Integrated | Integrated |
| aggregate and gel | separated | separated | | | | |

1. A method for producing a polymer aggregate, comprising:
a step for bringing a polymer solution containing a polymer substance and cations for gel formation into contact with an anion solution containing anions for gel formation to form a gel at an interface between the polymer solution and the anion solution; and
a step for causing the polymer substance to aggregate inside the gel.

2. The method for producing a polymer aggregate according to claim 1, wherein the cations for gel formation are at least one selected from multivalent cations or hydrogen ions.

3. The method for producing a polymer aggregate according to claim 1, wherein the anions for gel formation are alginate ions.

4. The method for producing a polymer aggregate according to claim 1, wherein the anion solution further contains an aggregation promoter for the polymer substance.

5. The method for producing a polymer aggregate according to claim 1, wherein the polymer solution further contains a dissolution promoter for the polymer substance.

6. The method for producing a polymer aggregate according to claim 1, wherein solvents of the polymer solution and the anion solution are water.

7. The method for producing a polymer aggregate according to claim 1, wherein the polymer substance is a protein.

8. The method for producing a polymer aggregate according to claim 7, wherein the protein is a polypeptide derived from a spider silk protein.
